(19) United States
(12) Patent Application Publication
(10) Pub. No.: US 2026/0162830 A1
(43) Pub. Date: Jun. 11, 2026

(54) REAL TIME IDENTIFICATION OF HIERARCHICAL CONDITION CATEGORIES IN ORDER TO PROVIDE ACCURATE RISK ADJUSTMENT OF PATIENTS AND METHODS

(71) Applicant: Retrieve Medical, Inc., Basking Ridge, NJ (US)

(72) Inventors: Donald C. Thomas, III, St Petersburg Beach, FL (US); Christopher E. Orlando, Verona, NJ (US)

(21) Appl. No.: 19/412,046

(22) Filed: Dec. 8, 2025

Related U.S. Application Data
(60) Provisional application No. 63/729,818, filed on Dec. 9, 2024.

(51) Int. Cl. G16H 50/30; G06F 40/289; G16H 10/60; G16H 15/00

(52) U.S. Cl. CPC G16H 50/30; G06F 40/289; G16H 10/60; G16H 15/00

(57) ABSTRACT

A system and method for real time identification of hierarchical condition categories for accurate risk adjustment of patients is provided for improving accuracy of risk adjustment factor scoring using structured and unstructured patient data. The system and method for real time identification of hierarchical condition categories may include a triage and assembly aspect, deep search and augmentation aspect, hierarchy and learning aspect, real-time validation aspect, and finalization aspect. A method for identifying hierarchical condition categories for improving accuracy of risk adjustment factor scoring is also provided.

### CROSS-REFERENCE TO RELATED APPLICATION

This application claims the priority from U.S. provisional patent application Ser. No. 63/729,818 filed Dec. 9, 2024. The foregoing application is incorporated in its entirety herein by reference.

### FIELD OF THE INVENTION

The present disclosure relates to a real time identification of hierarchical condition categories in order to provide accurate risk adjustment of patients. More particularly, the disclosure relates to identifying hierarchical condition categories indicative of improving accuracy of risk adjustment factor scoring.

### BACKGROUND

Care for non-hospitalized patients is compensated through payors that, using various methodologies, reimburse for that care based on the difficulty of delivering specific treatments to a specific patient. Patient care was traditionally paid almost anecdotally, based on what could be charged. This “fee for service” system was replaced years ago by a set of interrelated coding systems designed to standardize definitions for clinical care activities and procedures.

Diagnoses and treatments are identified very specifically. Diagnoses are commonly provided by ICD-10 codes and procedures by Current Procedural Terminology (CPT) Codes. ICD-10 codes define diagnoses in granular detail and likewise CPTs define very specific clinical activities. They can both be combined together in groups that are used to inform inpatient (DRGs) and outpatient (HCCs) documentation systems that ultimately define cost of care and billing. Final composites of either of the groupings are used to define visit difficulty and, from that, the payment for that specific visit.

There are differences between the way reimbursement is handled in outpatient settings versus inpatient ones. Both are calculated based on the interaction of patient illness burden, and the type (cost) of delivering care; inpatient visit Diagnosis-Related Groups (DRG) concentrate on factors which directly affect a particular care session as an inpatient episode, whereas HCCs concentrate on considerations which are factored into the patient's yearly health maintenance expense. The methodologies are not equivalent, nor can they be easily cross-translated. While there are relative similarities in the calculation of visit cost methodologies, the characteristics which define the issues that are relevant to patient care complexity are not the same for most patients in those very different clinical settings. Each unique visit has to be calculated individually, leading to overhead and inefficiency.

The documentation for each clinical interaction has to specifically justify each part of the patient's assessment at the time of record completion, but the volume and complexity of background, current and correlated material needed to correctly execute this task makes it virtually impossible for that to happen within the time allocated for a patient visit. This difficulty leads to either extensive extra work hours per patient (increased physician burnout), additional staff (financial problems), and/or limited documentation (introducing legal, financial, and accuracy problems).

In an ambulatory environment, when a patient record does not include proper Hierarchical Condition Categories (HCC's) including chronic conditions, the patient's risk score is not properly adjusted, and the providers'reimbursement is incomplete because the basis for cost of care determination is not based on a properly described patient. Additionally, there are multiple derivative assessments and forecasts which are improperly derived because of this flawed data.

The problem is not easily rectified—it takes time to research and document the information needed to inform these diagnostic descriptors, but patient flow requirements have decreased the available time resources available to the clinician in the face of these increased documentation time requirements. This leads to a classic Hobson's choice: inadequate documentation versus extra work outside of work.

Since doing nothing to solve the problem has been shown to be a major contributor to physician burnout problems, and negatively affects the viability of the patient care entity, there have been concerted efforts made in the current state of the art to provide solutions which mainly fall into two categories, both of which present new difficulties. For solutions which involve adding more staff, costs are prohibitive in many cases and create financial stress in most others. Solutions of the current state of the art may operate in editor mode, relying on retrospective record corrections which predefine the scope of the interaction and increase the potential for missed search parameters. Retrospective corrections necessitate rework (like queries), which increases physician work, burnout, and resistance. For solutions in the current state of the art which are automated, costs may be more reasonable, but the other two problems stated above continue to be unresolved and remain in some iteration.

Therefore, a need exists to solve the deficiencies present in the prior art. What is needed is a system to assist with identification of hierarchical condition categories. What is needed is a system to improve accuracy of determining risk adjustment factors of patients. What is needed is a system and method for detecting and recording hierarchical condition categories to comprehensively be included for a risk adjustment factor. What is needed is a system and method using machine learning and artificial intelligence to assist with identification of hierarchical condition categories. What is needed is a system and method capable of identifying hierarchical condition categories and providing accurate risk adjustment factors for patients in approximately real time. What is needed is a system and method to convert medical coding languages to be compliant with hierarchical condition category standards.

### SUMMARY

An aspect of the disclosure advantageously provides a system to assist with identification of hierarchical condition categories. An aspect of the disclosure advantageously provides a system to improve accuracy of determining risk adjustment factors of patients. An aspect of the disclosure advantageously provides a system and method for detecting and recording hierarchical condition categories to comprehensively be included for a risk adjustment factor. An aspect of the disclosure advantageously provides a system and method using machine learning and artificial intelligence to assist with identification of hierarchical condition categories. An aspect of the disclosure advantageously provides a system and method capable of identifying hierarchical condition categories and providing accurate risk adjustment factors for patients in approximately real time. An aspect of the disclosure advantageously provides a system and method to convert medical coding languages to be compliant with hierarchical condition category standards.

Accordingly, the disclosure may feature a system for real-time identification of hierarchical condition categories to assist with risk adjustment of patients, the system comprising a processor and a memory coupled to the processor, the memory storing instructions. When executed by the processor, the instructions may cause the system to access an electronic health record associated with a patient to retrieve a set of patient data, wherein the set of patient data includes structured data and unstructured clinical notes. The system may apply natural language processing to the unstructured clinical notes to extract one or more relevant medical terms. Additionally, the system may identify a set of potential hierarchical condition categories based on the structured data and the one or more relevant medical terms extracted from the unstructured clinical notes. The system may also generate a crosswalk between an identified medical coding language entry and a corresponding hierarchical condition category code. The system may calculate a risk adjustment factor score based on the corresponding hierarchical condition category code in substantially real-time during a patient encounter. Furthermore, the system may present the set of potential hierarchical condition categories and the risk adjustment factor score to a clinician via a user interface for validation.

In another aspect, the system may conduct a secondary data sweep of the set of patient data to identify and code an enumerated but previously uncoded item relevant to hierarchical condition category determination.

In another aspect, the system may utilize an artificial intelligence algorithm to create the crosswalk between a finding in the set of patient data and a rule set governing hierarchical condition category relevant statements. The artificial intelligence algorithm may be configured to self-learn from the validation provided by the clinician to improve accuracy of the crosswalk over time.

In another aspect, the medical coding language may include codes according to International Classification of Diseases, Tenth Revision (ICD-10).

In another aspect, the system may sort the set of patient data to remove a duplicate item and group a set of similar items to retain a highest value hierarchical condition category.

In another aspect, the system may update the electronic health record to reflect a validated hierarchical condition category determination following an interaction with the clinician.

In another aspect, the system may generate a comprehensive report based on the validation from the clinician to support a clinical decision or a billing process.

In another aspect, the set of patient data may further include at least one of a past medical history, a lab result, an imaging report, and/or a medication list.

Accordingly, the disclosure may feature a method for identifying and validating hierarchical condition categories in a healthcare setting. The method may include accessing, via a computing device, a medical record of a patient to retrieve relevant medical data. The method may additionally include analyzing the relevant medical data using natural language processing to identify a potential hierarchical condition category and a chronic condition, wherein analyzing includes extracting a medical phrase from an unstructured note. The method may include converting an identified International Classification of Diseases, Tenth Revision (ICD-10) code found within the relevant medical data to a corresponding hierarchical condition category code. Furthermore, the method may include calculating a risk adjustment factor score based on the corresponding hierarchical condition category code. The method may also include presenting the potential hierarchical condition category and supporting information to a clinician for review. The method may include updating the medical record with a validated hierarchical condition category following the review by the clinician.

In another aspect, the method may include conducting a search for chronic condition data within the medical record and, if present, integrating the chronic condition data into a search for the potential hierarchical condition category.

In another aspect, the method may include performing a secondary sweep of the medical record to detect an enumerated item that is relevant to the risk adjustment factor score but is uncoded.

In another aspect, the method may include sorting a collection of identified hierarchical condition categories into a hierarchical list and discarding a duplicate entry to retain a highest value hierarchical condition category.

In another aspect, calculating the risk adjustment factor score occurs in substantially real-time during a visit between the patient and the clinician.

In another aspect, the method may include calculating a reimbursement rate based on the validated hierarchical condition category and the risk adjustment factor score.

In another aspect, the method may include utilizing a machine learning algorithm trained on a dataset of medical records to identify and extract relevant medical information from the relevant medical data.

Accordingly, the disclosure may feature a method for improving accuracy of risk adjustment factor scoring using hierarchical condition coding. The method may include initiating a data collection process to gather patient-specific information from an electronic health record. Additionally, the method may include analyzing the patient-specific information using an artificial intelligence algorithm and natural language processing to identify a set of relevant health conditions. The method may also include mapping the set of relevant health conditions to a standardized medical code, wherein the standardized medical code includes a hierarchical condition category code. The method may include validating the set of relevant health conditions by cross-referencing against an established medical guideline. The method may further include displaying a result of the analyzing and the mapping to a clinician via an interface. The method may include receiving input from the clinician to confirm or modify the set of relevant health conditions. The method may be performed by an analytic engine operating in substantially real-time.

In another aspect, the method may include analyzing the patient-specific information includes identifying a risk factor that is not explicitly coded in the electronic health record.

In another aspect, the method may include generating a comprehensive report based on the input from the clinician to support a clinical decision or a billing process.

In another aspect, the artificial intelligence algorithm may be configured to identify a pattern or a correlation in the patient-specific information indicative of an underlying health issue affecting the risk adjustment factor scoring.

In another aspect, the method may include substantially automatically linking a finding from the natural language processing with an existing diagnosis to determine an appropriate hierarchical condition category code.

Terms and expressions used throughout this disclosure are to be interpreted broadly. Terms are intended to be understood respective to the definitions provided by this specification. Technical dictionaries and common meanings understood within the applicable art are intended to supplement these definitions. In instances where no suitable definition can be determined from the specification or technical dictionaries, such terms should be understood according to their plain and common meaning. However, any definitions provided by the specification will govern above all other sources.

Various objects, features, aspects, and advantages described by this disclosure will become more apparent from the following detailed description, along with the accompanying drawings in which like numerals represent like components.

### BRIEF DESCRIPTION OF THE DRAWINGS

FIG. 1 is a block diagram view of an illustrative system enabled by this disclosure.

FIG. 2 is a block diagram view of an illustrative computerized device, according to an embodiment of this disclosure.

FIG. 3 is a flow chart view of an illustrative operation of identifying hierarchical conditions, according to an embodiment of this disclosure.

FIG. 4 is a flow chart view of an illustrative operation of identifying and validating hierarchical condition categories in a healthcare setting, according to an embodiment of this disclosure.

FIG. 5 is a flow chart view of an illustrative operation of improving accuracy of risk adjustment factor scoring using hierarchical condition coding, according to an embodiment of this disclosure.

### DETAILED DESCRIPTION

The following disclosure is provided to describe various embodiments of a real time identification of hierarchical condition categories in order to provide accurate risk adjustment of patients. Skilled artisans will appreciate additional embodiments and uses of the present invention that extend beyond the examples of this disclosure. Terms included by any claim are to be interpreted as defined within this disclosure. Singular forms should be read to contemplate and disclose plural alternatives. Similarly, plural forms should be read to contemplate and disclose singular alternatives. Conjunctions should be read as inclusive except where stated otherwise.

Expressions such as “at least one of A, B, and C” should be read to permit any of A, B, or C singularly or in combination with the remaining elements. Additionally, such groups may include multiple instances of one or more element in that group, which may be included with other elements of the group. All numbers, measurements, and values are given as approximations unless expressly stated otherwise.

For the purpose of clearly describing the components and features discussed throughout this disclosure, some frequently used terms will now be defined, without limitation. The term hierarchical condition coding, as it is used throughout this disclosure, is defined as a system that categorizes patient diagnoses into groups with similar levels of complexity and cost, ensuring accurate risk adjustment and reimbursement for healthcare providers. The term risk adjustment factor, as it is used throughout this disclosure, is defined as a score used to predict healthcare costs by summarizing a patient's health status based on demographics and medical conditions. The term diagnosis related group, as it is used throughout this disclosure, is defined as a classification of hospital stays and/or patients into categories with similar diagnoses and treatments, allowing for standardized payments based on the expected resource consumption. The term ICD-10, as it is used throughout this disclosure, is defined as a standardized system used globally to classify and code diseases, symptoms, and causes of death for medical documentation, billing, and research, also known as International Classification of Diseases, Tenth Revision.

The term natural language processing, as it is used throughout this disclosure, is defined as an ability for computers to understand, interpret, and generate human language. The term machine learning, as it is used throughout this disclosure, is defined as a technique of operating computers to learn from data and make predictions or decisions without explicit programming. The term artificial intelligence, as it is used throughout this disclosure, is defined as an operation of computer systems that can learn from data and perform complex tasks, adapting their behavior and improving their performance over time. The term crosswalk, as it is used throughout this disclosure, is defined as a mapping or translation between different data formats or schemas, allowing for data exchange and interoperability between systems. The term real-time, as it is used throughout this disclosure, is defined as the processing of data or events as immediately as they occur as practically capable, without any intentional delay or buffering.

Various aspects of the present disclosure will now be described in detail, without limitation. In the following disclosure, a real time identification of hierarchical condition categories in order to provide accurate risk adjustment of patients will be discussed. Those of skill in the art will appreciate alternative labeling of the real time identification of hierarchical condition categories in order to provide accurate risk adjustment of patients as a hierarchical condition category identification system to improve accuracy of risk adjustment, risk adjustment factor accuracy improvement system using hierarchical condition coding, hierarchical condition coding identification system with risk adjustment factor accuracy improvement, the invention, or other similar names. Similarly, those of skill in the art will appreciate alternative labeling of the real time identification of hierarchical condition categories in order to provide accurate risk adjustment of patients as a method of improving risk adjustment for patients via identification of hierarchical condition categories, technique for improving record for risk adjustment factors using identified hierarchical condition categories in real time, method for identifying hierarchical condition categories in real time to improve accuracy of risk adjustment factor determiniation, method, operation, the invention, or other similar names. Skilled readers should not view the inclusion of any alternative labels as limiting in any way.

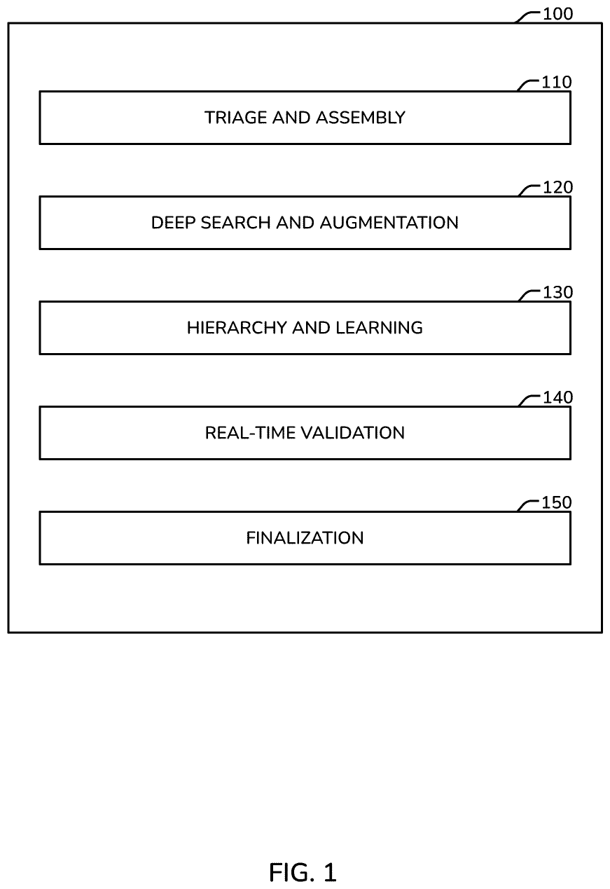

Referring now to FIGS. 1-5, the real time identification of hierarchical condition categories in order to provide accurate risk adjustment of patients will now be discussed in more detail. An illustrative system for real time identification of hierarchical condition categories in order to provide accurate risk adjustment of patients 100 may include a triage and assembly aspect 110, deep search and augmentation aspect 120, hierarchy and learning aspect 130, real-time validation aspect 140, finalization aspect 150, and additional components that will be discussed in greater detail below. The real time identification of hierarchical condition categories in order to provide accurate risk adjustment of patients may operate one or more of these components interactively with other components for identifying hierarchical condition categories indicative of improving accuracy of risk adjustment factor scoring.

The triage and assembly aspect 110 will now be discussed in greater detail. FIG. 1 highlights an example of the triage and assembly aspect, which may also be discussed along with other figures. This section covers the system overview, initiation of the encounter, and the initial collection and aggregation of patient data.

The following disclosure enables a system and corresponding method to, in substantially real-time and without intended delay, apply advanced Natural Language Processing (NLP) and AI augmentation within an analytical engine, for example, the Retrieve Dx engine. This facilitates the immediate identification and validation of Hierarchical Condition Categories (HCCs) by analyzing comprehensive patient data, enabling concurrent Risk Adjustment Factor (RAF) calculations and accurate reimbursement determinations during patient encounters. This automation, achieved through AI and NLP algorithms processing both structured and unstructured data, optimizes patient record management and reimbursement workflows, significantly reducing physician workload while enhancing documentation accuracy and completeness. Consequently, this invention improves both patient care outcomes and provider compensation by addressing beneficial inefficiencies in existing processes.

Using the Retrieve Dx engine, HCC's may be identified both beforehand and during the patient examination based on patient's records and medications. This identification may be accomplished, for example, using NLP and structured queries. AI augmentation and self-learning create increasingly efficient crosswalks between findings rule sets that govern HCC relevant statements. Thus, correct risk adjustment score may be determined through calculations based specifically on each unique patient encounter.

In one embodiment, during a first sweep of the analytic engine, for example Retrieve Dx engine, all or substantially all of the ICD-10 items are collected and converted to relevant HCC codes via a crosswalk. Then, additional sweeps may detect and collect enumerated items that are relevant but uncoded. The collected enumerated items may then be coded, for example, to relevant HCC codes. The contents of the data pull may be sorted and reassembled with duplicates discarded. A process may also be provided to put similar items together so that the ascendant or highest value HCC can be selected and retained according to coding rules.

According to an embodiment enabled by this disclosure, an analytic engine may be used, for example the Retrieve Dx engine, with NLP and AI augmentation to identify and validate HCCs in real-time during patient encounters. This process may include several key steps, some examples of which are provided below, without limitation.

To facilitate initiation of a patient encounter, an illustrative system may access electronic health records (EHR) and collect relevant patient data. This data may include demographics, medical history, previous diagnoses, treatments, medications, and lab results, without limitation. By aggregating this information, the system can create a comprehensive patient profile for review.

This automated data retrieval streamlines the clinical workflow and allows healthcare providers to focus on patient interaction rather than administrative tasks. This comprehensive data collection may help to ensure that the system has a complete picture of the patient's health status. This includes both structured data, such as lab values and coded diagnoses, and unstructured data, such as clinical notes and discharge summaries. By accessing and analyzing this diverse information, the system can more accurately identify and validate HCCs for improved risk adjustment and reimbursement.

The deep search and augmentation aspect 120 will now be discussed in greater detail. FIG. 1 highlights an example of the deep search and augmentation aspect, which may also be discussed along with other figures. This section covers the analysis of unstructured data (NLP), mapping of codes, secondary sweeps for uncoded items, and specific examples of identifying complex conditions.

NLP analysis may be performed on the retrieved data, such as analyzing clinical notes and structured data to extract relevant medical terms and potential HCCs. This analysis may involve, for example and without limitation, identifying key phrases associated with specific conditions, like “chronic heart failure” or “type 2 diabetes,” within the text of a doctor's notes. Furthermore, the NLP analysis may recognize and interpret medical terminology and abbreviations commonly used in clinical documentation, ensuring accurate identification of potential HCCs. This automated extraction process reduces the manual effort required for reviewing patient records and improves the efficiency of HCC coding.

Using the results of the NLP analysis, an initial HCC identification may be provided by mapping ICD-10 codes to HCC codes and categorizing risk factors. For example, a mapping operation may include linking the extracted medical terms to their corresponding ICD-10 codes and then using established guidelines to map those codes to the appropriate HCC categories. For example, if the NLP analysis identifies the term “congestive heart failure,” the system may map this to the corresponding ICD-10 code and then determine the appropriate HCC category for heart failure. This automated mapping process facilitates accurate and efficient HCC identification based on the patient's medical information.

Secondary data collection may be performed, which may include conducting additional data sweeps to identify and code uncoded items. This step helps ensure that relevant patient information is captured, including conditions or procedures that may have been missed during the initial data extraction. For instance, the system might review physician notes or lab reports for any undocumented diagnoses or treatments that could impact risk adjustment. This thorough approach improves the accuracy and completeness of the patient's medical record, ultimately leading to more precise HCC coding, RAF score determination, and appropriate reimbursement.

In some embodiments, chronic conditions may be crosswalked to HCC code, which may be used to calculate a RAF score, which may further be used to determine a payment reimbursement from Medicaid, Medicare, and/or other payor for care relating to a patient. For example, automated crosswalking of ICD-10 codes to HCC codes may utilize advanced NLP and machine learning algorithms trained on vast datasets of medical records, enabling them to identify and extract relevant medical information from both structured and unstructured data sources, such as clinical notes and lab results. The NLP engine may analyze the text, identify key medical terms, and map them to corresponding ICD-10 codes. These codes may then be processed by a machine learning model trained on established HCC coding guidelines, enabling the system to accurately assign the appropriate HCC category based on the patient's diagnoses and risk factors. This automated process may help to ensure accurate and efficient HCC coding, facilitating real-time risk adjustment and reimbursement calculations.

In one example, provided to clearly disclose an embodiment enabled by this disclosure and without limitation, a patient with a history of type 2 diabetes, coded as E11.9 in ICD-10, may visit their physician. The system enabled by this disclosure, equipped with NLP capabilities, may analyze the physician's notes from the encounter. The system may identify the ICD-10 code and perform the crosswalk, recognizing that E11.9 maps to HCC 19-Diabetes without Complication. However, the NLP engine and/or analytic engine such as Retrieve Dx, may also detect the phrase “patient reports numbness and tingling in feet” within the clinical notes. Based on its training data, the system of this example may recognize this as a potential indicator of diabetic polyneuropathy. The system may then automatically link this finding with the existing diabetes diagnosis and determine that a more specific HCC code, HCC 18-Diabetes with Chronic Complications, is more appropriate. This information, along with the supporting evidence from the clinical notes, is then presented to the physician in a user-friendly interface for review and confirmation. The physician can then validate the HCC coding, ensuring accurate documentation and appropriate risk adjustment for the patient.

The hierarchy and learning aspect 130 will now be discussed in greater detail. FIG. 1 highlights an example of the hierarchy and learning aspect, which may also be discussed along with other figures. This section covers data deduplication, sorting for the highest value HCC, and the use of AI for continuous learning and improvement.

Data may then be stored such that duplication may be detected and removed, which may include sorting data, grouping similar items, and discarding duplicates. Data deduplication advantageously improves operation, as each piece of patient information may be uniquely represented. Data deduplication additionally reduces the opportunity for redundant data to influence the analysis. For example, if a patient has multiple instances of the same diagnosis code recorded, the system may identify and remove the duplicates, preventing that condition from being overweighted in the risk adjustment calculation. This data cleaning step may thus improve the accuracy and reliability of the HCC coding process.

The cleaned data may be applied to AI augmentation and self-learning, which may enhance accuracy and efficiency of the data processing through AI algorithms and self-learning. For example, a system enabled by this disclosure may utilize the refined data to train its AI models, allowing them to identify patterns and make more accurate predictions about HCCs. For example, the AI algorithms may learn to recognize subtle relationships between different diagnoses or identify potential risk factors that might be easily missed or overlooked by human reviewers. In at least one embodiment, this continuous learning process may include a feedback loop, which may allow the system to adapt and improve its performance over time, leading to more efficient and accurate HCC coding.

The real-time validation aspect 140 will now be discussed in greater detail. FIG. 1 highlights an example of the real-time validation aspect, which may also be discussed along with other figures. This section covers the interaction with the clinician during the patient visit, presenting findings, and the interface used for confirmation.

The selected solutions may be presented for physician review and validation for each HCC determination in real-time. This interaction may occur during part of the visit between a patient and their physician and precedes record closure for the visit. Relevant information needed to support this validation is displayed during the encounter, eliminating most rework (queries), and drastically speeding up documentation time while dramatically increasing accurate identification of patient conditions.

Real-time HCC determination may then be provided, for example, by performing real-time calculations and dynamic interaction with the physician. For example, a system enabled by this disclosure may, without intended delay, analyze patient data and present potential HCCs to the physician during the encounter, facilitating immediate review and validation. This real-time feedback loop greatly increases the likelihood that the HCC coding is accurate and complete, allowing for prompt risk adjustment and appropriate reimbursement. Furthermore, this dynamic interaction may enable physicians to address any discrepancies or provide additional information that may impact the HCC coding, improving the overall quality of patient care and documentation.

The physician may be provided with a user-friendly interface for real-time review and validation of HCCs generated using the operations described above. For example, an interface might present the identified HCCs in a clear and concise manner, allowing the physician to quickly assess their accuracy and completeness. The physician could then interact with the interface to confirm or modify the HCC assignments, ensuring that the final coding accurately reflects the patient's health status. This streamlined review process advantageously reduces the administrative burden on physicians, prioritizes the physician's time for providing medical care, and promotes accurate documentation for improved patient care and appropriate reimbursement.

The finalization aspect 150 will now be discussed in greater detail. FIG. 1 highlights an example of the finalization aspect, which may also be discussed along with other figures. This section covers record closure, final risk adjustment calculations, reimbursement, and reporting on outcomes and benefits.

After performance of the real-time validation aspect, the validated data may undergo record closure, where the patient records may be updated and documentation finalized. For example, a thorough review of the collected data and HCC assignments may be performed to ensure accuracy and completeness. Any discrepancies or missing information can be addressed, and the final validated data may then be integrated into the patient's electronic health record. The procedure of this example ensures that the patient's medical record accurately reflects their current health status and supports proper billing and reimbursement for the provided care.

Reimbursement calculation may then be performed with improved accuracy by calculating reimbursement rates based on validated RAF scores. This advantageously increases the likelihood that healthcare providers receive appropriate compensation for the complexity of the patients they treat. By accurately capturing the patient's health status through HCC coding and RAF score calculation, a system enabled by this disclosure promotes fair and equitable reimbursement for healthcare services. This, in turn, supports the financial stability of healthcare providers, allowing them to continue delivering quality care to their patients.

Enhanced documentation and reporting may then be generated, which may include comprehensive reports to support clinical decisions, billing, and quality of care provided. For example, reports could provide a detailed overview of the patient's health status, including their diagnosed conditions, treatment history, and risk factors. This information may be used to inform clinical decision-making, support accurate billing and coding, improve quality ratings, and track the overall quality of care provided. Additionally, these reports may be used to identify areas for improvement in patient care and to monitor the effectiveness of interventions.

Building upon the concept of HCC coding, the RAF score is used to estimate the healthcare resources required for a patient. This score, which may be calculated based on demographics, clinical conditions, and HCCs, acts as a risk profile for individuals enrolled in some healthcare plans, for example, Medicare Advantage plans. As will be appreciated by those of skill in the art, a score below 1.0 represents the average population, while higher scores indicate greater complexity and the need for more resources. By accurately identifying and coding HCCs, as described previously, a system enabled by this disclosure advantageously increases the likelihood that the RAF score adequately reflects the patient's health status. This may lead to appropriate reimbursement from Medicare and/or other payors, enabling healthcare providers to deliver comprehensive care and effectively manage the patient's conditions. This process, conducted in real-time during patient encounters, seamlessly integrates into the clinical workflow without disruption, ultimately improving both patient care and provider compensation.

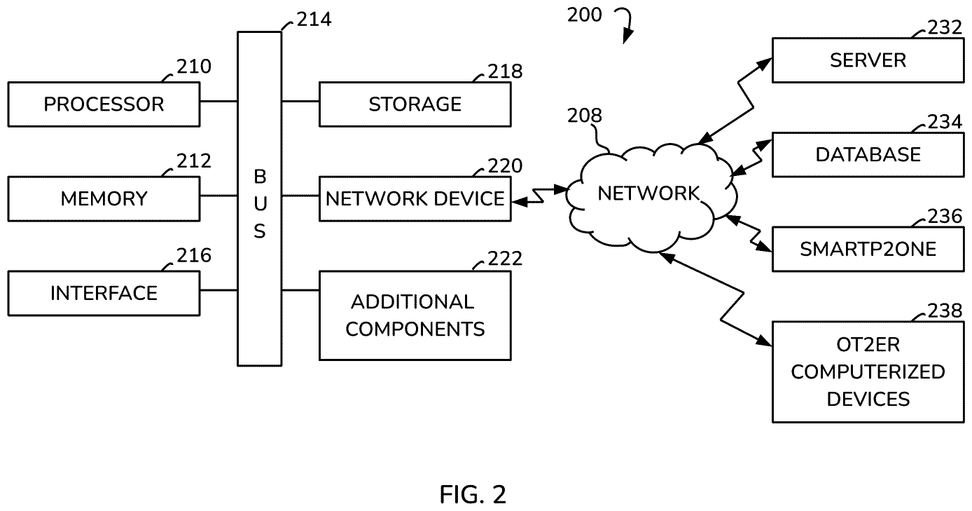

Referring now to FIG. 2, an illustrative computerized device 200 will now be discussed in greater detail, without limitation. The computerized device may include a processor 210, memory 212, network controller 220, and optionally an input/output (I/O) controller 216. Skilled artisans will appreciate additional embodiments of a computerized device that may omit one or more of the aforementioned components or include additional components 222 without limitation. The processor 210 may receive and analyze data. The memory 212 may store data, which may be used by the processor 210 to perform the analysis. The memory 212 may also receive data indicative of results from the analysis of data by the processor 210.

The memory 212 may include volatile memory modules, such as random-access memory (RAM), or non-volatile memory modules, such as flash-based memory. Skilled artisans will appreciate storage devices 218 may be provided, such as, for example, mechanical hard drives, solid state data, and removable storage devices.

The computerized device may also include a network controller 220. The network controller 220 may receive data from other components of the computerized device to be communicated with other computerized devices via a network 208. The communication of data may be performed wirelessly. More specifically, without limitation, the network controller 220 may communicate and relay information from one or more components of the computerized device, or other devices and/or components connected to the computerized device, to additional connected devices. Connected devices are intended to include data servers 232, database 234, additional computerized devices 238, mobile computing devices, smart phones 236, tablet computers, and other electronic devices that may communicate digitally with another device. In one example, the computerized device may be used as a server 232 to analyze and communicate data between connected devices.

The computerized device may also include an I/O interface 216. The I/O interface 216 may be used to transmit data between the computerized device and extended devices. Examples of extended devices may include, but should not be limited to, a display, external storage device, human interface device, printer, sound controller, or other components that would be apparent to a person of skill in the art. Additionally, one or more of the components of the computerized device may be communicatively connected to the other components via the I/O interface 216.

The components of the computerized device may interact with one another via a bus 214. Those of skill in the art will appreciate various forms of a bus 214 that may be used to transmit data between one or more components of an electronic device, which are intended to be included within the scope of this disclosure. The computerized device may communicate with one or more connected devices via a network 208. The computerized device may communicate over the network 208 by using its network controller 220. More specifically, the network controller 220 of the computerized device may communicate with the network controllers of the connected devices. The network 208 may be, for example, the internet. As another example, the network 208 may be a WLAN. However, skilled artisans will appreciate additional networks to be included within the scope of this disclosure, such as intranets, local area networks, wide area networks, peer-to-peer networks, and various other network formats. Additionally, the computerized device and/or connected devices may communicate over the network 208 via a wired, wireless, or other connection, without limitation.

In operation, a method may be provided for identifying hierarchical condition categories for improving accuracy of risk adjustment factor scoring. Those of skill in the art will appreciate that the following methods are provided to illustrate an embodiment of the disclosure and should not be viewed as limiting the disclosure to only those methods or aspects. Skilled artisans will appreciate additional methods within the scope and spirit of the disclosure for performing the operations provided by the examples below after having the benefit of this disclosure. Such additional methods are intended to be included by this disclosure.

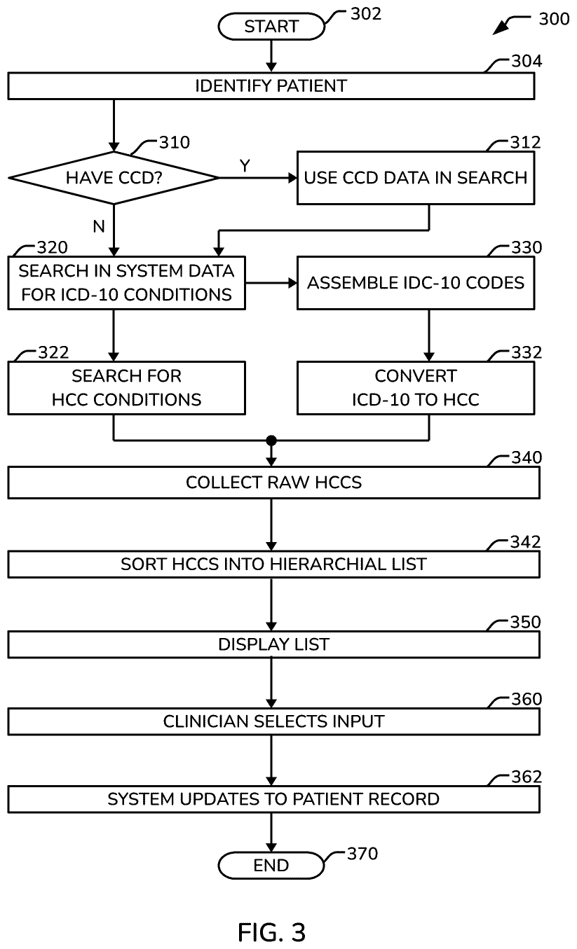

Referring now to flowchart 300 of FIG. 3, an example method for an illustrative operation of identifying hierarchical conditions, according to an embodiment of this disclosure will be described, without limitation. Process 300 is designed to operate in substantially real-time, allowing for the integration of risk adjustment factor scoring directly into the clinical workflow. The operation may begin at Step 302, which may be triggered by the opening of a patient chart or the commencement of a patient encounter.

At Step 304, the system may proceed to identify the patient. This step involves accessing an electronic health record associated with the patient to retrieve a set of patient data. This step may also include verifying the patient's identity to ensure that the correct records are being accessed. Relevant historical data may be retrieved, forming the basis for subsequent analyses. This data may include previous diagnoses, treatments, lab results, imaging reports, and current medications. Accurate patient identification may ensure that all available patient information is collected for a comprehensive assessment.

Once the patient is identified, the process moves to a decision node at Step 310, where the system determines if the patient has existing Chronic Condition Data (CCD). This step queries the historical records to see if there are long-standing diagnoses that are relevant to the risk adjustment factor accuracy improvement engine. This may involve examining the patient's records for any previously documented chronic conditions that could influence the risk adjustment factor. Chronic conditions are relevant for accurate risk adjustment as they impact the patient's overall health status, and the complexity of care required.

If the determination is affirmative (indicating “Y”), the process follows the path to Step 312. If the determination is negative (indicating “N”), the process may bypass the integration of pre-existing CCD and proceed directly to scanning current system data at Step 320.

At Step 312, if CCD is present, the system is configured to use the CCD data in the search. This involves retrieving the established chronic conditions and preparing them for integration with new data. By using CCD data, the system ensures that the “search” for risk factors is informed by the patient's longitudinal history, rather than just the acute presentation of the current visit. The CCD information may be used to enhance the identification of relevant HCCs, ensuring that the risk adjustment calculations are as accurate as possible and may involve incorporating the CCD information with current visit data to enhance the identification of relevant HCCs and ensure comprehensive risk adjustment.

Following Step 312, or directly from Step 310 if no CCD is found, the process proceeds to Step 320, which involves searching in system data for ICD-10 conditions. This step represents a broad sweep of the available electronic health records to locate items already coded in the ICD-10 format. The search in Step 320 may access various components of the set of patient data. This may include structured data fields where diagnoses have been formally entered, as well as other repositories within the system data where ICD-10 codes may be stored or referenced, such as previous billing claims or problem lists.

From Step 320, the workflow may branch into parallel or simultaneous operations to ensure comprehensive coverage. One path leads to Step 330, while another path leads to Step 322, allowing the system to handle explicitly coded data and uncoded potential conditions concurrently. For example, if the patient does not have a CCD, the system may perform a search within the patient's data to identify any ICD-10 conditions. This search may involve extracting diagnoses and conditions coded using the ICD-10 system. The system may scan through the patient's clinical notes, lab results, and other medical records to gather relevant ICD-10 codes. This process may ensure that even if CCD is not available, pertinent medical conditions are identified and considered for risk adjustment.

At Step 330, the system functions to assemble the identified ICD-10 codes. This assembly process aggregates the codes found during the search in Step 320, creating a consolidated list of the patient's current coded diagnoses. This list serves as the raw material for the subsequent translation into risk adjustment categories.

Simultaneously or sequentially, at Step 322, the system performs a search for HCC conditions. Unlike the search for explicit ICD-10 codes, this step may involve analyzing the relevant medical data to identify potential hierarchical condition categories that are not yet coded. The search at Step 322 may utilize natural language processing (NLP) to analyze unstructured clinical notes. By extracting relevant medical terms from narrative text, the system can identify a risk factor or a condition that implies a hierarchical condition category, even if an ICD-10 code has not yet been assigned.

This deep search at Step 322 effectively functions as a secondary data sweep. It allows the system to detect an enumerated but previously uncoded item relevant to hierarchical condition category determination, ensuring that the risk profile is not under-represented due to documentation gaps. A thorough search may be conducted to identify all possible HCC conditions. This search may involve using NLP to analyze unstructured data and identify potential HCCs that may not be explicitly coded. The NLP technology may scan through clinical notes, physician comments, and other unstructured data sources to extract relevant medical terms and phrases that could indicate additional HCCs. This step may ensure that all relevant conditions are identified, even those not directly coded as ICD-10 or HCC.

Following the assembly of codes at Step 330, the process moves to Step 332, where the system operates to convert ICD-10 to HCC. This step involves generating a crosswalk between the identified medical coding language entry (the ICD-10 code) and a corresponding hierarchical condition category code. All or substantially all identified ICD-10 codes may be assembled from the patient's records. This step may include collecting diagnoses from various sources such as clinical notes, lab results, imaging reports, and current medications. The assembly of ICD-10 codes may form a comprehensive list of the patient's medical conditions. This list may be used for mapping these conditions to HCC codes in the next step. The system may ensure that no relevant ICD-10 codes are overlooked, providing a complete picture of the patient's health status.

The conversion at Step 332 relies on established mapping guidelines. The system applies these rules to translate the granular diagnosis codes into the broader categories used for calculating the risk adjustment factor score, ensuring regulatory compliance and accuracy. The outputs from the conversion at Step 332 and the direct search for conditions at Step 322 converge at Step 340. Here, the system functions to collect raw HCCs. This step may aggregate potential risk categories identified through both the direct code conversion and the NLP-driven search. This conversion process may involve mapping each ICD-10 code to the appropriate HCC, ensuring that relevant diagnoses are accurately represented in the HCC system. This step may be used for risk adjustment calculations as HCC codes are used to determine the patient's overall health risk and the associated reimbursement rates. The system may use established mapping guidelines to ensure accurate and consistent conversion of ICD-10 to HCC codes.

At Step 340, the collection includes the “raw” data, which may contain duplicates or conflicting categories. For example, a patient might have an ICD-10 code for diabetes and a text note indicating a diabetic complication, resulting in two related but distinct potential HCCs. The system may collect raw HCCs identified during the initial and secondary data sweeps. This collection may include HCCs derived from both structured data (e.g., ICD-10 codes) and unstructured data (e.g., clinical notes). The system may ensure that substantially all potential HCCs are captured, providing a comprehensive list of risk factors for the patient. This step may be used for ensuring that the risk adjustment factor accurately reflects the patient's health status and the complexity of their care.

The process proceeds to Step 342, which involves sorting the HCCs into a hierarchical list. This sorting process applies the logic of the “hierarchy” inherent in the coding system, where certain severe conditions supersede less severe manifestations of the same disease process. During Step 342, the system may sort the set of patient data to remove duplicate items. It may also group a set of similar items to ensure that only the most appropriate category is retained for the final calculation, preventing “double dipping” in the risk score while ensuring the highest relevant complexity is captured.

The sorting at Step 342 ensures that the system retains a highest value hierarchical condition category. This logic is beneficial for calculating an accurate risk adjustment factor score that reflects the true burden of illness without inflating the score through redundancy. The identified HCCs may be sorted into a hierarchical list according to coding rules. This may involve grouping similar conditions together and ensuring that the highest value HCCs are selected and retained based on their impact on the risk adjustment factor. The sorting process may follow established guidelines to prioritize HCCs that have the most significant effect on the patient's risk score. This step may ensure that the final list of HCCs accurately reflects the patient's health risk and the complexity of their care needs.

Once the list is sorted and refined, the process moves to Step 350, where the system operates to display the list. This display is presented to a clinician via a user interface, bringing the computational insights into the visible workflow of the provider. The display at Step 350 may present the set of potential hierarchical condition categories and supporting information. This transparency allows the clinician to see not just the suggested code, but the evidence (such as a specific note or lab result) that triggered the suggestion.

For example, the sorted list of HCCs may be displayed to the clinician. This display may include detailed information about each identified HCC, providing the clinician with a comprehensive overview of the patient's risk factors. The interface may present the HCCs in a user-friendly manner, allowing the clinician to review and understand the risk factors quickly. This step may be used for ensuring that the clinician has all the necessary information to validate the identified HCCs and make informed decisions about the patient's care.

Moving to Step 360, the process requires interaction where the clinician selects inputs. This step represents the validation phase, where the human expert reviews the system-generated list to confirm or modify the set of relevant health conditions. At Step 360, the clinician may check off valid conditions, reject incorrect findings, or add additional context. The system is configured to receive this input to confirm the accuracy of the automated analysis. This interaction at Step 360 may also enable the system to utilize an artificial intelligence algorithm to self-learn. By observing which suggestions the clinician accepts or rejects, the system can refine its crosswalks and search parameters to improve the accuracy of the crosswalk over time.

The clinician may review the displayed list of HCCs and select the relevant inputs for each patient encounter. This step may involve validating the identified HCCs and making any necessary adjustments based on clinical judgment. The clinician can add, modify, or remove HCCs as needed to ensure that the final list accurately reflects the patient's health status. This interaction may ensure that the risk adjustment calculations are based on accurate and validated data.

Following the clinician's selection, the process proceeds to Step 362, where the system updates to the patient record. This step commits the validated hierarchical condition categories to the electronic health record, finalizing the documentation for the encounter. The update at Step 362 ensures that the medical record reflects the validated hierarchical condition category determination. This updated record then serves as the basis for calculating the final risk adjustment factor score and generating the appropriate reimbursement rate. The system may update the patient's record with the validated HCCs selected by the clinician. This update may ensure that the patient's record accurately reflects relevant risk factors and diagnoses. The system may finalize the documentation for the current visit, incorporating the validated HCCs into the patient's permanent medical record.

By automating the search and conversion while reserving Step 360 for human validation, the process 300 balances computational efficiency with clinical accuracy. It reduces the administrative burden on the clinician while ensuring that the accurate documentation is complete, which is beneficial for accurate reimbursement and ongoing patient care. With the validated HCC data, the system may additionally calculate the appropriate reimbursement rates based on the validated RAF score. This calculation may involve using the validated HCCs to determine the accurate cost and complexity of patient care. The reimbursement algorithms may ensure that providers are compensated accurately for the services provided, reflecting the true cost and effort involved in patient care. This step may ensure that the healthcare provider receives fair and appropriate compensation for the care delivered to the patient.

The system may also generate comprehensive documentation and detailed reports based on the validated data. This documentation may ensure high-quality patient records, better statistical analysis, and improved quality ratings for the healthcare provider. The enhanced documentation may include substantially all necessary information to support clinical decisions, billing, and quality reporting. This step may contribute to better overall patient care and operational efficiency, ensuring that all relevant information is accurately captured and reported.

Finally, the process concludes at Step 370. At this stage, the real-time identification loop is complete for the specific patient encounter, and the system may be ready to re-initiate the process at Step 302 for the next patient.

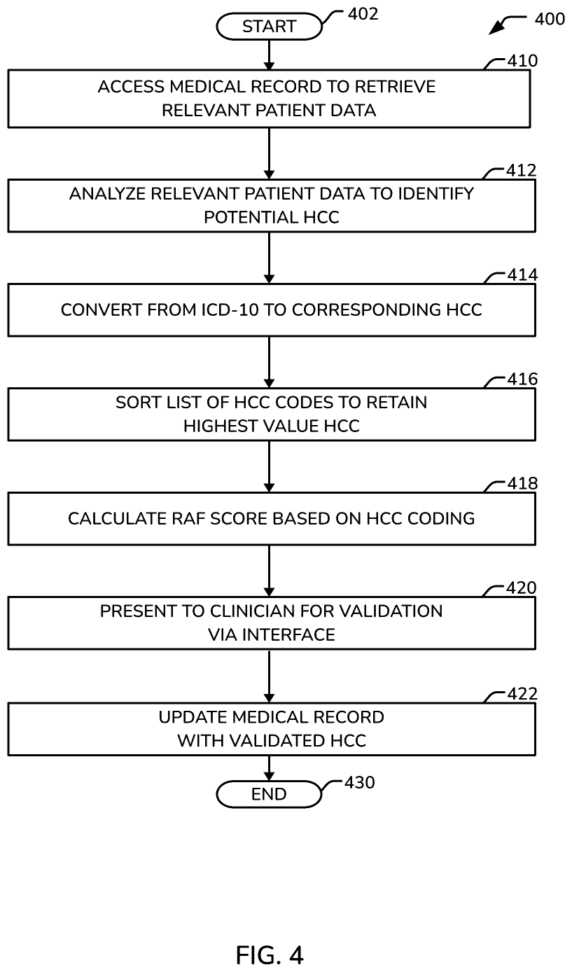

Referring now to flowchart 400 of FIG. 4, an example method for an illustrative operation of identifying and validating hierarchical condition categories in a healthcare setting will be described, without limitation. Starting with Block 402, the operation may begin by initiating an analytic engine or computing device designed to assist with risk adjustment factor scoring. This initiation may trigger a sequence of steps designed to process patient data in substantially real-time.

At Step 410, the method may include accessing a medical record of a patient to retrieve relevant medical data. This step serves as the foundation for the analysis, ensuring that the system has access to a comprehensive set of information regarding the patient's health history and current status. The relevant medical data may include both structured data fields and unstructured narrative text. The retrieval of relevant medical data at Step 410 may further involve conducting a search for chronic condition data within the medical record. If such chronic condition data is present, the method may involve integrating the chronic condition data into a subsequent search for a potential hierarchical condition category. This ensures that historical conditions that persist are not overlooked during the current encounter.

Once the data is accessed, the method proceeds to Step 412, which may involve analyzing the relevant medical data to identify a potential hierarchical condition category. This analysis acts as a deep search of the patient's record to surface conditions that affect the complexity of care. This identification process may operate on both the structured codes already present and the unstructured text that has not yet been codified. During the analysis at Step 412, the method may utilize natural language processing to identify the potential hierarchical condition category and a chronic condition. Natural language processing allows the system to interpret human language found in clinical documentation, bridging the gap between free-text descriptions and standardized medical coding.

For example, the step of analyzing may include extracting a medical phrase from an unstructured note. For example, a clinician's note might describe symptoms or conditions in a narrative format that does not immediately correspond to a code. The method is capable of parsing this unstructured note to find medical phrases that indicate a risk-adjustable condition. To enhance the accuracy of this analysis, the method may further comprise utilizing a machine learning algorithm trained on a dataset of medical records. This machine learning algorithm may be configured to identify and extract relevant medical information from the relevant medical data, leveraging patterns learned from vast amounts of historical data to improve detection rates. In addition to the primary analysis, the method may include performing a secondary sweep of the medical record. This secondary sweep may be designed to detect an enumerated item that is relevant to the risk adjustment factor score but is uncoded. This feature advantageously captures data points that may have been enumerated in a list or table but failed to be translated into a claimable code.

Following the identification of potential conditions, the method may proceed to Step 414, which involves converting an identified ICD-10 code found within the relevant medical data to a corresponding hierarchical condition category code. This conversion creates a bridge between the diagnosis codes used for standard billing and the risk adjustment categories used for value-based care. This conversion process helps ensure that the specificities of the ICD-10 coding system are accurately translated into the broader categories required for risk adjustment. The system may effectively “crosswalk” the granular diagnosis data into the appropriate risk category, ensuring the patient's complexity is properly represented. After conversion, the method may move to Step 416, which involves sorting the list of HCC codes. Because a patient may have multiple conditions that map to related categories, or multiple instances of the same category, organization of the data is necessary for accurate scoring.

For example, Step 416 may include sorting a collection of identified hierarchical condition categories into a hierarchical list. This organization reflects the “hierarchy” in Hierarchical Condition Categories, where more severe manifestations of a disease may supersede less severe ones for scoring purposes. As part of this sorting process, the method may further include discarding a duplicate entry to retain a highest value hierarchical condition category. This ensures that the risk score is not artificially inflated by counting the same condition multiple times and that the most significant contributor to the patient's risk is the one preserved for calculation.

Once the list is sorted and refined, the method proceeds to Step 418, which may involve calculating a risk adjustment factor score based on the corresponding hierarchical condition category code. This calculation aggregates the weights of the validated HCCs to produce a numeric value representing the patient's health status. This calculation of the risk adjustment factor score may occur in substantially real-time during a visit between the patient and the clinician. By performing this calculation during the patient encounter, the system allows the clinician to understand the impact of the documentation on the risk profile while the patient is still present. Furthermore, the method may include calculating a reimbursement rate based on the validated hierarchical condition category and the risk adjustment factor score. This financial calculation links the clinical documentation directly to the resources allocated for the patient's care, ensuring appropriate compensation for the complexity of the case.

At Step 420, the method involves presenting the potential hierarchical condition category and supporting information to a clinician for review. This presentation may occur via a user interface that integrates into the clinician's workflow, displaying the findings of the NLP and AI analysis clearly. The step of presenting allows for human-in-the-loop validation. The clinician can review the supporting information extracted from the unstructured notes to confirm that the identified potential HCC is accurate and currently relevant to the patient. Following the review by the clinician, the method proceeds to Step 422, which involves updating the medical record with a validated hierarchical condition category. This step commits the findings to the official record, transforming the “potential” categories into “validated” data points that become part of the patient's history.

Finally, the method 400 may conclude at Step 430. By completing this flow, the system ensures that the patient's medical record is comprehensive, accurate, and properly coded for risk adjustment, reducing the need for retrospective queries and administrative rework.

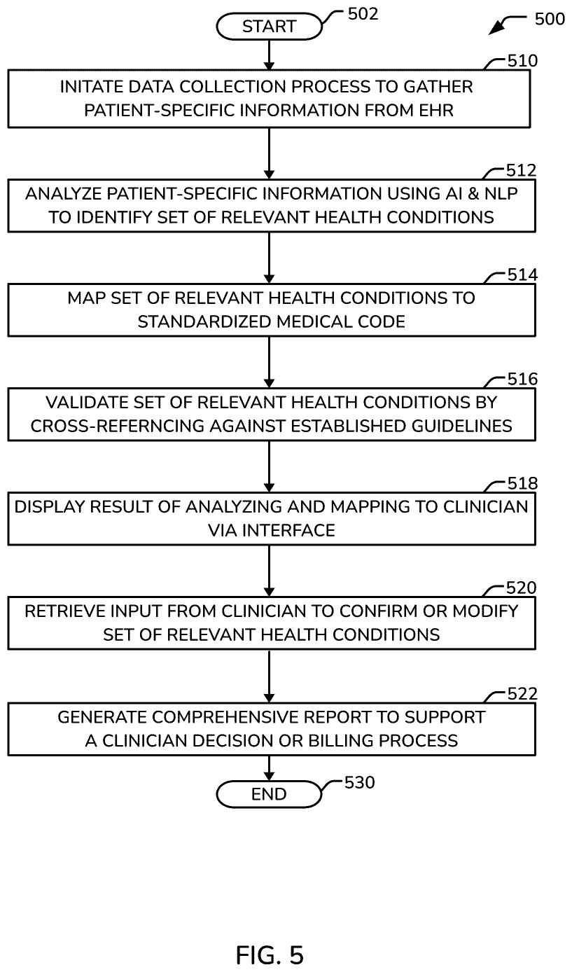

Referring now to flowchart 500 of FIG. 5, an example method for an illustrative operation of improving accuracy of risk adjustment factor scoring using hierarchical condition coding will be described, without limitation. Starting with Block 502, the operation may begin by signaling the activation of an analytic engine configured to assist healthcare providers. The method 500 is preferably performed by an analytic engine operating in substantially real-time. This ensures that the insights generated by the system are available to the clinician during the patient encounter, rather than requiring retrospective review. By operating without significant delay, the system integrates seamlessly into the clinical workflow.

At Step 510, the method includes initiating a data collection process to gather patient-specific information from an electronic health record. This step involves pulling a comprehensive set of data points associated with the patient to build a foundation for analysis. The system accesses the electronic health record to retrieve the necessary history and current status of the patient. The patient-specific information gathered at Step 510 is not limited to simple demographic data but includes a rich history of care. This collection ensures that the subsequent analysis is based on a complete view of the patient, including past diagnoses, treatments, and ongoing conditions that may be relevant to risk adjustment.

Proceeding to Step 512, the method involves analyzing the patient-specific information using an artificial intelligence algorithm and natural language processing to identify a set of relevant health conditions. This step represents a sophisticated examination of the data, going beyond simple keyword matching to understand the context of the patient's health. During this analysis, the system may utilize natural language processing to parse unstructured text. This allows the method to include identifying a risk factor that is not explicitly coded in the electronic health record. For example, symptoms or social determinants of health mentioned in free-text notes can be identified as relevant health conditions.

Furthermore, the analysis at Step 512 may involve the use of an artificial intelligence algorithm configured to identify a pattern or a correlation in the patient-specific information. The AI may detect subtle indicators in the data that suggest an underlying health issue affecting the risk adjustment factor scoring, even if that issue has not yet been formally diagnosed. This deep analysis allows the system to surface a set of relevant health conditions that might otherwise be missed. By identifying these patterns and correlations, the analytic engine can propose conditions that more accurately reflect the complexity of the patient's health status, thereby improving the accuracy of the eventual risk score. Once the conditions are identified, the method moves to Step 514, which involves mapping the set of relevant health conditions to a standardized medical code. This translation step is useful for ensuring that the clinical findings are compatible with billing and reporting systems.

For example, the standardized medical code mapped during Step 514 includes an HCC code. The system correlates between clinical descriptions and the specific coding language required for risk adjustment models, such as those used by Medicare. As part of this mapping process, the method may further comprise automatically linking a finding from the natural language processing with an existing diagnosis to determine an appropriate hierarchical condition category code. For instance, the system might link a new symptom found in the notes with a chronic condition in the history to suggest a code reflecting a higher level of severity or complication.

After mapping, the method proceeds to Step 516, which involves validating the set of relevant health conditions by cross-referencing against an established medical guideline. This automated validation step acts as a quality control measure, ensuring that the suggested codes are clinically appropriate and meet the criteria for documentation. This cross-referencing helps prevent coding errors by checking the identified conditions against rules and standards. The system ensures that the evidence found in the patient-specific information is sufficient to support the assignment of the standardized medical code according to the established guidelines.

At Step 518, the method involves displaying a result of the analyzing and the mapping to a clinician via an interface. The interface serves as the point of interaction between the analytic engine and the human provider, presenting the complex findings in a clear and actionable format. The display at Step 518 presents the set of relevant health conditions and the proposed codes in a manner that facilitates quick review. Because the method operates in substantially real-time, this information is presented to the clinician while they are assessing the patient, allowing for verification during the patient encounter.

Following the display, the method moves to Step 520, which includes receiving input from the clinician to confirm or modify the set of relevant health conditions. This step ensures that the final record reflects the clinician's professional judgment. The clinician can accept the system's suggestions or make adjustments based on their direct observation of the patient. This interaction is a beneficial component of the validation process. By retrieving input from the clinician, the system ensures that the “relevant health conditions” are not just statistical probabilities but are clinically confirmed diagnoses that belong in the patient's medical record.

Once the clinician has provided input, the method proceeds to Step 522, which involves generating a comprehensive report. This report consolidates the findings, the codes, and the validation actions into a final document for the encounter. The comprehensive report generated at Step 522 is designed to support a clinical decision or a billing process. By capturing the validated health conditions and their corresponding HCC codes, the report provides the necessary documentation to justify the risk adjustment factor score and the associated reimbursement.

Finally, the method 500 concludes at Step 530. Through this process, the system successfully transforms raw patient data into accurate, validated, and coded information that supports both high-quality patient care and accurate financial risk adjustment.

While various aspects have been described in the above disclosure, the description of this disclosure is intended to illustrate and not limit the scope of the invention. The invention is defined by the scope of the appended claims and not the illustrations and examples provided in the above disclosure. Skilled artisans will appreciate additional aspects of the invention, which may be realized in alternative embodiments, after having the benefit of the above disclosure. Other aspects, advantages, embodiments, and modifications are within the scope of the following claims.

What is claimed is:

1. A system for real-time identification of hierarchical condition categories to assist with risk adjustment of patients, the system comprising a processor and a memory coupled to the processor, the memory storing instructions that, when executed by the processor, cause the system to:
access an electronic health record associated with a patient to retrieve a set of patient data, wherein the set of patient data includes structured data and unstructured clinical notes;
apply natural language processing to the unstructured clinical notes to extract one or more relevant medical terms;
identify a set of potential hierarchical condition categories based on the structured data and the one or more relevant medical terms extracted from the unstructured clinical notes;
generate a crosswalk between an identified medical coding language entry and a corresponding hierarchical condition category code;
calculate a risk adjustment factor score based on the corresponding hierarchical condition category code in substantially real-time during a patient encounter; and
present the set of potential hierarchical condition categories and the risk adjustment factor score to a clinician via a user interface for validation.

2. The system of claim 1, wherein the instructions further cause the system to conduct a secondary data sweep of the set of patient data to identify and code an enumerated but previously uncoded item relevant to hierarchical condition category determination.

3. The system of claim 1, wherein the instructions further cause the system to utilize an artificial intelligence algorithm to create the crosswalk between a finding in the set of patient data and a rule set governing hierarchical condition category relevant statements; and
wherein the artificial intelligence algorithm is configured to self-learn from the validation provided by the clinician to improve an accuracy of the crosswalk over time.

4. The system of claim 1, wherein the medical coding language comprises codes according to International Classification of Diseases, Tenth Revision (ICD-10).

5. The system of claim 1, wherein the instructions further cause the system to sort the set of patient data to remove a duplicate item and group a set of similar items to retain a highest value hierarchical condition category.

6. The system of claim 1, wherein the instructions further cause the system to update the electronic health record to reflect a validated hierarchical condition category determination following an interaction with the clinician.

7. The system of claim 1, wherein the instructions further cause the system to generate a comprehensive report based on the validation from the clinician to support a clinical decision or a billing process.

8. The system of claim 1, wherein the set of patient data further comprises at least one of a past medical history, a lab result, an imaging report, or a medication list.

9. A method for identifying and validating hierarchical condition categories in a healthcare setting, the method comprising:
accessing, via a computing device, a medical record of a patient to retrieve relevant medical data;
analyzing the relevant medical data using natural language processing to identify a potential hierarchical condition category and a chronic condition, wherein analyzing includes extracting a medical phrase from an unstructured note;
converting an identified International Classification of Diseases, Tenth Revision (ICD-10) code found within the relevant medical data to a corresponding hierarchical condition category code;
calculating a risk adjustment factor score based on the corresponding hierarchical condition category code;
presenting the potential hierarchical condition category and supporting information to a clinician for review; and
updating the medical record with a validated hierarchical condition category following the review by the clinician.

10. The method of claim 9, further comprising conducting a search for chronic condition data within the medical record and, if present, integrating the chronic condition data into a search for the potential hierarchical condition category.

11. The method of claim 9, further comprising performing a secondary sweep of the medical record to detect an enumerated item that is relevant to the risk adjustment factor score but is uncoded.

12. The method of claim 9, further comprising sorting a collection of identified hierarchical condition categories into a hierarchical list and discarding a duplicate entry to retain a highest value hierarchical condition category.

13. The method of claim 9, wherein calculating the risk adjustment factor score occurs in substantially real-time during a visit between the patient and the clinician.

14. The method of claim 9, further comprising calculating a reimbursement rate based on the validated hierarchical condition category and the risk adjustment factor score.

15. The method of claim 9, further comprising utilizing a machine learning algorithm trained on a dataset of medical records to identify and extract relevant medical information from the relevant medical data.

16. A method for improving accuracy of risk adjustment factor scoring using hierarchical condition coding, the method comprising:
initiating a data collection process to gather patient-specific information from an electronic health record;
analyzing the patient-specific information using an artificial intelligence algorithm and natural language processing to identify a set of relevant health conditions;
mapping the set of relevant health conditions to a standardized medical code, wherein the standardized medical code includes a hierarchical condition category code;
validating the set of relevant health conditions by cross-referencing against an established medical guideline;
displaying a result of the analyzing and the mapping to a clinician via an interface; and
receiving input from the clinician to confirm or modify the set of relevant health conditions;
wherein the method is performed in substantially real-time.

17. The method of claim 16, wherein analyzing the patient-specific information includes identifying a risk factor that is not explicitly coded in the electronic health record.

18. The method of claim 16, further comprising generating a comprehensive report based on the input from the clinician to support a clinical decision or a billing process.

19. The method of claim 16, wherein the artificial intelligence algorithm is configured to identify a pattern or a correlation in the patient-specific information indicative of an underlying health issue affecting the risk adjustment factor scoring.

20. The method of claim 16, further comprising automatically linking a finding from the natural language processing with an existing diagnosis to determine an appropriate hierarchical condition category code.
